(19) United States
(12) Patent Application Publication
(10) Pub. No.: US 2015/0351634 A1
(43) Pub. Date: Dec. 10, 2015

(54) Method for Evaluating the Potential of a Test Composition to Inhibit Demineralization or Promote Remineralization of Enamel

(71) Applicant: COLGATE-PALMOLIVE COMPANY, New York, NY (US)

(72) Inventors: Aarti Rege, East Windsor, NJ (US); Richard Sullivan, Atlantic Highlands, NJ (US)

(73) Assignee: Colgate-Palmolive Company, New York, NY (US)

(21) Appl. No.: 14/761,548

(22) PCT Filed: Jan. 18, 2013
(86) PCT No.: PCT/US2013/022128

§ 371 Date: Jul. 16, 2015

(51) Int. Cl. A61B 5/00; A61K 8/19; A61K 8/44; A61Q 11/00; A61C 19/06; A61K 8/24

(52) U.S. Cl. CPC A61B 5/0071; A61B 5/0088; A61C 19/066; A61K 8/24; A61K 8/44; A61Q 11/00; A61K 8/19

(57) ABSTRACT

In situ methods of evaluating the potential of a test composition to inhibit demineralization of the tooth enamel and/or to promote remineralization of acid-softened enamel using quantitative light fluorescence (QLF), e.g., by using an intra-oral appliance comprising tooth substrates to permit application of test composition to the substrate when in the mouth and erosive challenge when outside the mouth, and measuring the degree of mineralization or demineralization of the tooth substrate by in the presence and absence of treatment with the test composition.

### BACKGROUND OF THE INVENTION

Dental erosion involves demineralization and damage to the tooth structure due to acid attack from nonbacterial sources. Erosion is found initially in the enamel and, if unchecked, may proceed to the underlying dentin. Dental erosion may be caused or exacerbated by acidic foods and drinks, exposure to chlorinated swimming pool water, and regurgitation of gastric acids. The tooth enamel is a negatively charged surface, which naturally tends to attract positively charged ions such as hydrogen and calcium ions, while resisting negatively charged ions such as fluoride ions. Depending upon relative pH of surrounding saliva, the tooth enamel will lose or gain positively charged ions such as calcium ions. Generally saliva has a pH between 7.2 to 7.4. When the pH is lowered and concentration of hydrogen ions becomes relatively high, the hydrogen ions will replace the calcium ions in the enamel, forming hydrogen phosphate (phosphoric acid), which damages the enamel and creates a porous, sponge-like roughened surface. If saliva remains acidic over an extended period, then remineralization may not occur, and the tooth will continue to lose minerals, causing the tooth to weaken and ultimately to lose structure.

There are various techniques available to measure and study dental erosion either directly or indirectly. Erosion is generally assessed by studying the changes in hard tissue structure or by measuring the loss of hard tissue on the surface. Approaches include chemical analysis of the dissolved minerals in an erosive solution, weight loss of a specimen exposed to an erosive solution, scanning electron microscopy (SEM), and surface profilometry. Current techniques have various disadvantages, e.g. being unsuitable for clinical use to measure erosion in situ, measuring erosion only in the late stages of the process when the physical structure of the tooth is lost, and/or potentially destroying the delicate structure of the demineralized tooth in the course of carrying out the measurement.

Quantitative Light-induced Fluorescence (QLF) is a visible light fluorescence that is used to detect early carious lesions and longitudinally monitor the progression or regression. In general, the strong light scattering in the lesion leads to shorter light path than in sound enamel, and the fluorescence becomes weaker. see Karlsson et al., “Supplementary Methods for Detection and Quantification of Dental Caries:, J. Laser Dent., vol. 16(1): 6-14 (2008).

The area of demineralization can be quantified and its progress monitored. Sound, healthy tooth enamel yields a higher intensity of fluorescence under excitation from some wavelengths than does de-mineralized enamel that has been damaged by caries infection. For example, blue laser or high intensity LED light will make healthy teeth auto-fluoresce in the yellow-green range. Areas that have lost mineral have lower fluorescence and appear darker in comparison to a sound tooth surface. The intensity of the fluorescence can be measured quantitatively, and the correlation between mineral loss and loss of fluorescence for blue light excitation can be used to identify and assess carious areas of the tooth.

A different relationship has been found for red light excitation, a region of the spectrum for which bacteria and bacterial by-products in carious regions absorb and fluoresce more pronouncedly than do healthy areas. Software is used to quantify the fluorescence from a white spot or the area/volume associated with the lesion. Generally, subjects with existing white spot lesions are recruited as panelists. The measurements are performed in vivo with real teeth. The lesion area/volume is measured at the beginning of the clinical. The reduction (improvement) in lesion area/volume is measured at the end of 6 months of product use. The data is often reported as a percent improvement versus baseline.

### BRIEF SUMMARY OF THE INVENTION

The invention provides the use of QLF to measure and track erosion in situ. QLF is found to accurately depict damage due to dental erosion at a very early stage, when demineralization has begun but before there has been an actual loss of structure.

The invention provides methods of evaluating the ability of a test composition to protect against demineralization and/or to remineralize acid-softened enamel.

In some embodiments, subjects are asked to wear intra-oral appliances to contain tooth substrates that can be exposed to acid challenges and product treatments in a regimented way that is representative of daily habits. Once the acid exposure/treatment portion of the study is complete the tooth substrates can be retrieved from the appliance and analyzed ex vivo. This type of methodology offers the advantages of avoiding inducing in vivo erosion in subjects taking part in the study (which can be ethically problematic), and the possibility to perform extensive analyses on the retrieved substrates which could otherwise not be carried out in the oral cavity.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DESCRIPTION OF THE INVENTION

The following description of the preferred embodiment(s) is merely exemplary in nature and is in no way intended to limit the invention, its application, or uses.

In one embodiment, the invention provides a method of evaluating the potential of a test composition to inhibit demineralization of the tooth enamel and/or to promote remineralization of acid-softened enamel (Method 1), the method comprising the steps of:
(i) placing the intra-oral appliance in the mouth of a test subject, wherein the intra-oral appliance comprises a tooth substrate; and one or more of the following steps comprising in any order,
(ii) treating part or all of the tooth substrate with the test composition, e.g., toothpaste or mouthwash, while the intra-oral appliance is in the mouth;
(iii) removing the intra-oral appliance from the mouth of a test subject;
(iv) exposing part or all of the tooth substrate to an erosive challenge, e.g. acid;
(v) maintaining the intra-oral appliance in the mouth of the test subject;
(vi) quantifying the degree of mineralization of the tooth substrate as a function of the degree of fluorescence of the tooth substrate upon exposure to blue laser light, wherein the degree of mineralization is positively correlated with the degree of fluorescence upon exposure to blue laser light, e.g., using quantitative light-induced fluorescence (QLF); and
(vii) the test composition is selected for use in a method to inhibit or reduce demineralization of the tooth enamel and/or to promote remineralization of acid-softened enamel if the test composition causes more fluorescence of the tooth substrate in the yellow-green light range than another test composition.

Other embodiments of the method include, but are not limited to:
1.1. The method may be in situ or in vitro nature of Method 1 and may apply to any or all of the individual steps of Method 1.
1.2. The tooth substrate may be an enamel specimen with dentin that fluoresces (sources include, but are not limited to naturally occurring enamel speciments humans, bovines, rodents, etc. or synthetically produced enamel specimens).
1.3. Method 1 wherein the test composition is a toothpaste, which is applied to all or part of the tooth substrate by brushing with water while the intra-oral appliance is in the mouth.
1.4. Method 1 wherein the test composition is a mouthwash, which is applied to all or part of the tooth substrate by rinsing the substrate with the mouthwash while the intra-oral appliance is in the mouth.
1.5. Any of the foregoing methods wherein the erosive challenge comprises exposure to acid media, e.g. pH 3-4, e.g. citric acid, for one or more periods of up to 20 minutes, e.g., 30 seconds to 10 minutes.
1.6. Any of the foregoing methods wherein part of the tooth substrate is shielded from exposure to erosive challenge and/or from treatment with the test composition, e.g., to provide a control.
1.7. Any of the foregoing methods wherein the tooth substrate is exposed to erosive challenge while outside of the mouth, then part of the tooth substrate is exposed to test composition while in the mouth and part is shielded from the test composition, and then the degree of mineralization of the tooth substrate is quantified, wherein a greater degree of mineralization in the part of the tooth substrate exposed to the test composition than the part shielded from test composition indicates that the test composition is effective to provide remineralization of the tooth substrate.
1.8. Any of the foregoing methods 1-1.4 wherein part of the tooth substrate is exposed to test composition while in the mouth and part is shielded from exposure to test composition, then the tooth substrate is exposed to erosive challenge while outside of the mouth, and the degree of mineralization of the tooth substrate is quantified again, wherein a greater degree of mineralization in the part of the tooth substrate exposed to the test composition than the part shielded from test composition indicates that the test composition is effective to inhibit demineralization of the tooth substrate.
1.9. Any of the foregoing methods 1-1.4 wherein part of the tooth substrate is shielded both from both treatment with test composition and from exposure to erosive challenge, wherein the difference in mineralization between the part of the tooth substrate exposed to erosive challenge and treatment with test composition and the part of the tooth substrate shielded from erosive challenge and treatment with test composition provides a measure of the potential of the test composition to inhibit demineralization of the tooth enamel and/or to promote remineralization of acid-softened enamel each of the tested formulations provided.
1.10. Any of the foregoing methods 1-1.4 wherein the test composition selected for use in a method to inhibit or reduce demineralization of the tooth enamel and/or to promote remineralization of acid-softened enamel contains one or more of arginine, fluoride, bi-valent metal ion (e.g. stannous, zinc or calcium) or potassium.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

Embodiments of the present invention are further described in the following examples. The examples are merely illustrative and do not in any way limit the scope of the invention as described and claimed.

### EXAMPLE

### In Situ Assays

Methodology

### Enamel Protection Assay

This assay measures the ability of test products to protect against demineralization. The polished pristine enamel surface of bovine dental substrates is half-masked with acid resistance adhesive tape to ensure partial protection from acid exposure. The substrates are then evaluated for five consecutive days during which they are exposed to four (4) daily erosive challenges each 10 min in duration and outside the oral cavity. At the beginning of each day before the first erosive challenge, and after the last erosive challenge of the day subjects would brush with the assigned test product while wearing the appliance, therefore, exposing the tooth substrates to the treatment itself. The daily treatment/challenge sequence can therefore be summarized as follows:
T-C-C-C-C-T
   where T=1 min brushing +1 min swishing with slurry, and C=10 min citric acid (pH=3.8). This particular design may be used to substantiate a claim of “enamel protection” because it affords the measurement of enamel loss reduction as caused by the test treatment on pristine enamel.

### Enamel Restoration/Remineralization Assay

This assay measures a product's ability to remineralize acid-softened enamel. In this design, each enamel substrate is initially softened by a 30 sec exposure to a 5% citric acid solution, and half-masked as described above prior to the start of the study. The substrates are then evaluated for one day during which the panelists performed two treatments with the assigned test product, and allowed for four (4) hours of intra-oral remineralization in between treatment events. The sequence for this type of design can therefore be summarized as follows:
T-4 hours-T-4 hours
   where,
T=1 min brushing +1 min swishing with slurry
4 hours =intra-oral remineralization

This particular design may be used to substantiate a claim of “enamel restoration/remineralization” because it affords the measurement of enamel loss reduction as caused by the test treatment on acid softened enamel.

### Substrate Analysis

QLF image analysis is employed as the end point measurement to quantify the extent of erosion induced on tooth substrates by the demineralization procedure described above. The analysis entails the following steps:

Substrate Preparation:

After removal from the intra-oral retainers, the tooth substrates are untaped, washed with DI water and allowed to air-dry for 15 min. Superficial deposits are removed by lightly polishing (5 rotations per substrate) on velvet scratch pad.

QLF Measurements:

All QLF images of tooth substrates are captured using a HV-F31F CCD camera (Hitachi, Japan) integrated into a sample chamber which prevented interference from surrounding light sources and is equipped with a z-translation stage. To quantify the naturally occurring fluorescence of the dentinal layer, the substrates are exposed to light from a blue LED ring illuminator. The image is filtered through a 520 nm high pass filter. As a result the camera is only exposed to wavelengths that exceed 520 nm. The substrate position (i.e. distance from camera) as well as illuminator intensity are adjusted to provided optimum illumination, and, once set, are kept constant from sample to sample. This is achieved by using a piece of fluorescent paper of defined size as a calibration sample, so that the same level of illumination is repeatedly obtained by adjusting the illuminator intensity and the distance from camera (i.e. z-translation).

Image Analysis:

All images are captured and analyzed with Imagepro 7 software (Microsoft) by a single operator blinded to sample identification. The average fluorescence intensity of each tooth substrate is measured from the taped (protected) and exposed (unprotected) sides individually. The average percent fluorescence (mineral) loss (Av %F) between the two sides is calculated according to equation 1:

Av%F=[Av(AΔunprotected-ΔF protected)/ΔF protected]* 100 Eq. 1

where ΔF is the difference in naturally occurring fluorescence to due to dentinal layer of the tooth substrate before and after the demineralization/treatment procedure. Once calculated, the change in average percent fluorescence of each substrate is interpreted as a function of mineral loss, therefore, affording a measure of how much anti-erosive protection each of the tested formulations provided.

### Example 1

### Comparison of Arginine/Fluoride vs. Fluoride Only Dentifrice

In this example, the effects of a toothpaste containing 8% by weight arginine and 1450 ppm fluoride (as monofluorophosphate (MFP)) in a calcium carbonate base was compared against another toothpaste containing 1450 ppm fluoride (as monofluorophosphate (MFP)) in a silica base was studied on a population of 24 adult males and/or females, ages 18 to 70.

### Test Protocol

The design of the study involved randomized, crossover investigation of two fluoride containing dentifrice products in a double-blind fashion over two testing periods using an intra-oral retainer model and measured the ability of the test product to prevent mineral loss from four times daily erosive challenge over a five day period. A one-week (−1/+3days) washout period preceded each one-week treatment phase.

Each subject was randomly assigned to a test product to be used. The panelists wore an intra-oral appliance fitted with three sound bovine enamel disks over a period of 5 days. Treatment during testing period involved dipping the appliance in a 1% citric acid (pH=3.8) solution four (4) times per day and brushing the teeth with appliance in the mouth with a randomly assigned dentifrice for a period of one minute two times per day (morning and evening) followed by swishing for 1 minute with the slurry.

After five days, the intra-oral appliance and specimens were collected from the panelist for measurements.

The main end point measurement was performed by Quantitative Light Fluorescence (QLF) technique. A relative loss of fluorescence was used as a means of quantifying mineral content. A paired t-test was conducted to determine if significant differences exist between the treatments.

### Results

The QLF data revealed an average % fluorescence (mineral) loss for the arginine/fluoride toothpaste was only 9.74% whereas the loss of fluorescence for fluoride toothpaste was 18.36% (higher percentages reflecting greater mineral loss in the tooth enamel). Statistical analysis showed that the observed differences were highly significant (p<0.001).

As those skilled in the art will appreciate, numerous changes and modifications may be made to the embodiments described herein without departing from the spirit of the invention. It is intended that all such variations fall within the scope of the appended claims.

We claim:

1. A method of evaluating the potential of a test composition to inhibit demineralization of the tooth enamel and/or to promote remineralization of acid-softened enamel, the method comprising the steps of:
(i) placing an intra-oral appliance in the mouth of a test subject, wherein the intra-oral appliance comprises a tooth substrate;
(ii) treating part or all of the tooth substrate with the test composition, e.g., toothpaste or mouthwash, while the intra-oral appliance is in the mouth;
(iii) removing the intra-oral appliance from the mouth of a test subject;
(iv) exposing part or all of the tooth substrate to an erosive challenge, e.g. acid;
(v) maintaining the intra-oral appliance in the mouth of the test subject;
(vi) quantifying the degree of mineralization of the tooth substrate as a function of the degree of fluorescence of the tooth substrate upon exposure to blue laser light, wherein the degree of mineralization is positively correlated with the degree of fluorescence upon exposure to blue laser light, using quantitative light-induced fluorescence (QLF); and
the test composition is selected for use in a method to inhibit or reduce demineralization of the tooth enamel and/or to promote remineralization of acid-softened enamel if the test composition causes more fluorescence of the tooth substrate in the yellow-green light range than another test composition.

2. The method of claim I wherein part of the tooth substrate is exposed to test composition while in the mouth and part is shielded from exposure to test composition, then the tooth substrate is exposed to erosive challenge while outside of the mouth, and the degree of mineralization of the tooth substrate is quantified again, wherein a greater degree of mineralization in the part of the tooth substrate exposed to the test composition than the part shielded from test composition indicates that the test composition is effective to inhibit demineralization of the tooth substrate.

3. A method of evaluating the potential of a test composition to inhibit demineralization of the tooth enamel and/or to promote remineralization of acid-softened enamel, the method comprising the steps of:
(i) placing an intra-oral appliance in the mouth of a test subject, wherein the intra-oral appliance comprises a tooth substrate;
(ii) removing the intra-oral appliance from the mouth of a test subject;
(iii) exposing part or all of the tooth substrate to an erosive challenge, e.g. acid;
(iv) maintaining the intra-oral appliance in the mouth of the test subject;
(v) treating part or all of the tooth substrate with the test composition, e.g., toothpaste or mouthwash, while the intra-oral appliance is in the mouth;
(vi) quantifying the degree of mineralization of the tooth substrate as a function of the degree of fluorescence of the tooth substrate upon exposure to blue laser light, wherein the degree of mineralization is positively correlated with the degree of fluorescence upon exposure to blue laser light, using quantitative light-induced fluorescence (QLF); and
(vii) the test composition is selected for use in a method to inhibit or reduce demineralization of the tooth enamel and/or to promote remineralization of acid-softened enamel if the test composition causes more fluorescence of the tooth substrate in the yellow-green light range than another test composition.

4. The method of claim 3, wherein the tooth substrate is exposed to erosive challenge while outside of the mouth, then part of the tooth substrate is exposed to test composition while in the mouth and part is shielded from the test composition, and then the degree of mineralization of the tooth substrate is quantified, wherein a greater degree of mineralization in the part of the tooth substrate exposed to the test composition than the part shielded from test composition indicates that the test composition is effective to provide remineralization of the tooth substrate.

5. The method of claim 1, wherein the test composition is a toothpaste, which is applied to all or part of the tooth substrate by brushing with water while the intra-oral appliance is in the mouth.

6. The method of claim 1, wherein the test composition is a mouthwash, which is applied to all or part of the tooth substrate by rinsing the substrate with the mouthwash while the intra-oral appliance is in the mouth.

7. The method of claim 1 wherein the erosive challenge comprises exposure to acid.

8. The method of claim 1 wherein part of the tooth substrate is shielded from exposure to erosive challenge and/or from treatment with the test composition.

9. The method of claim 1, wherein part of the tooth substrate is shielded both from both treatment with test composition and from exposure to erosive challenge, wherein the difference in mineralization between the part of the tooth substrate exposed to erosive challenge and treatment with test composition and the part of the tooth substrate shielded from erosive challenge and treatment with test composition provides a measure of the potential of the test composition to inhibit demineralization of the tooth enamel and/or to promote remineralization of acid-softened enamel each of the tested formulations provided.

10. The method of claim 1, wherein the test composition selected for use in a method to inhibit or reduce demineralization of the tooth enamel and/or to promote remineralization of acid-softened enamel contains one or more of arginine, fluoride, a bivalent metal ion or potassium.

11. A method of evaluating the potential of a test composition to inhibit demineralization of the tooth enamel and/or to promote remineralization of acid-softened enamel, the method comprising the steps of:
(i) placing an intra-oral appliance in the mouth of a test subject, wherein the intra-oral appliance comprises a tooth substrate;
(ii) treating part or all of the tooth substrate with the a toothpaste or mouthwash, while the intra-oral appliance is in the mouth;
(iii) removing the intra-oral appliance from the mouth of a test subject;
(iv) exposing part or all of the tooth substrate to an acid challenge;
(v) maintaining the intra-oral appliance in the mouth of the test subject;
(vi) removing the intra-oral appliance from the mouth of a test subject;
(vii) quantifying the degree of mineralization of the tooth substrate as a function of the degree of fluorescence of the tooth substrate upon exposure to blue laser light, wherein the degree of mineralization is positively correlated with the degree of fluorescence upon exposure to blue laser light, using quantitative light-induced fluorescence (QLF); and
(viii) the test composition is selected for use in a method to inhibit or reduce demineralization of the tooth enamel and/or to promote remineralization of acid-softened enamel if the test composition causes more fluorescence of the tooth substrate in the yellow-green light range than another test composition.

12. The method of claim 11, wherein the test composition selected for use in a method to inhibit or reduce demineralization of the tooth enamel and/or to promote remineralization of acid-softened enamel contains one or more of arginine, fluoride, a bivalent metal ion or potassium.

13. The method of claim 12, wherein the bi-valent metal ion is calcium, stannous, zinc or combinations thereof.

14. The method of claim 11, wherein the test composition contains arginine.

15. The method of claim 11, wherein the test composition contains fluoride.

16. The method of claim 11, wherein the test composition contains calcium, stannous, zinc or combinations thereof.

17. The method of claim 11, wherein the test composition contains potassium.
